(19) United States
(12) Patent Application Publication
(10) Pub. No.: US 2024/0058568 A1
(43) Pub. Date: Feb. 22, 2024

(54) Methods And Apparatuses For Generating Sleeping Aid Audio

(71) Applicant: Anhui Huami Health Technology Co., Ltd., Hefei, Anhui FTZ (CN)

(72) Inventors: Cong Zhang, Hefei, Anhui FTZ (CN); Zi Meng, Hefei, Anhui FTZ (CN); Guokang Zhu, Hefei, Anhui FTZ (CN); Yi Yu, Hefei, Anhui FTZ (CN)

(21) Appl. No.: 18/499,549

(22) Filed: Nov. 1, 2023

Related U.S. Application Data
(63) Continuation of application No. PCT/CN2022/104139, filed on Jul. 6, 2022.

(30) Foreign Application Priority Data
Aug. 25, 2021 (CN) 202110984332.8

(51) Int. Cl. A61M 21/02; G06F 3/16

(52) U.S. Cl. CPC A61M 21/02; G06F 3/165; A61M 2021/0027

(57) ABSTRACT

A method, an apparatus, a computer device and storage media for generating a sleeping aid audio are provided. The method for generating a sleeping aid audio includes: a plurality of sleeping aid audio spectrums are obtained; the plurality of sleeping aid audio spectrums are processed with a genetic algorithm, to obtain a sleeping aid audio spectrum chain; and the sleeping aid audio is generated according to the sleeping aid audio spectrum chain.

### CROSS-REFERENCE TO RELATED APPLICATION(S)

The present application is a continuation of International Application No. PCT/CN2022/104139, filed on Jul. 6, 2022, which claims priority and benefit of Chinese Patent Application No. 202110984332.8, filed Aug. 25, 2021, the entire disclosures of both of which are hereby incorporated by reference.

### TECHNICAL FIELD

Implementations of the present disclosure relate to the field of computer technologies, in particularly relate to methods, apparatuses, computer devices and storage medium for generating sleeping aid audio.

### BACKGROUND

Sleep can not only eliminate fatigue and create new vitality, but also improve immunity and disease resistance. Good immunity comes from good sleep. Music, as a multimedia carrier, is easy to be obtained and accepted, and has become primary means for sleeping aid. Music-based sleeping aid belongs to the category of psychological intervention, and aims to reduce stress and obtain physical and mental relaxation to achieve an effect of sleeping aid.

The sleeping aid audio used in related technologies is mostly created manually. However, the emotion expressed by this kind of sleeping aid audio is influenced by musicians' subjective ideas, and this kind of sleeping aid audio has a limited effect and application scope. Therefore, it remains a challenge how to generate sleeping aid audio scientifically and effectively.

### SUMMARY

The present disclosure provides a method, an apparatus, a non-transitory computer storage medium and a computer program product for generating sleeping aid audio.

According to a first aspect of the present disclosure, a method for generating sleeping aid audio is provided, and includes:

obtaining a plurality of sleeping aid audio spectrums;

processing the plurality of sleeping aid audio spectrums with a genetic algorithm, to obtain a sleeping aid audio spectrum chain; and

generating the sleeping aid audio according to the sleeping aid audio spectrum chain.

In some implementations, obtaining a plurality of sleeping aid audio spectrums includes:

obtaining m reference audio spectrums;

processing each reference audio spectrum with an identification model generated by training, to determine a sleeping aid value of the each reference audio spectrum; and

determining n reference audio spectrums from the m reference audio spectrums as the plurality of sleeping aid audio spectrums, in which each of the n reference audio spectrum corresponds to a sleeping aid value greater than a first threshold, m is greater than n and n is a natural number greater than 1.

In some implementations, processing the plurality of sleeping aid audio spectrums with the genetic algorithm, to obtain the sleeping aid audio spectrum chain includes:

processing the plurality of sleeping aid audio spectrums with the genetic algorithm based on the sleeping aid values corresponding to the plurality of sleeping aid audio spectrums, to obtain the sleeping aid audio spectrum chain.

In some implementations, processing the plurality of sleeping aid audio spectrums with the genetic algorithm based on the sleeping aid values corresponding to the plurality of sleeping aid audio spectrums, to obtain the sleeping aid audio spectrum chain includes:

selecting one or more target sleeping aid audio spectrums to be processed from the plurality of sleeping aid audio spectrums based on the sleeping aid values corresponding to the plurality of sleeping aid audio spectrums;

processing the one or more target sleeping aid audio spectrums by performing at least one of a crossover operation and a mutation operation, to generate a plurality of first-child-generation sleeping aid audio spectrums;

selecting one or more target first-child-generation sleeping aid audio spectrums from the plurality of first-child-generation sleeping aid audio spectrums based on the sleeping aid values corresponding to the plurality of first-child-generation sleeping aid audio spectrums; and

preforming, repeatedly until a number of operations performed reaches a preset value, at least one of the crossover operation and the mutation operation based on the one or more target first-child-generation sleeping aid audio spectrums, and

determining the sleeping aid audio spectrum chain according to the one or more target sleeping aid audio spectrums and the generated respective generations of target sleeping aid audio spectrums.

In some implementations, after the generating sleeping aid audio, the method further includes:

obtaining a sleep state of a user while a sleeping aid audio being played;

determining the sleeping aid value of the sleeping aid audio according to the sleep state; and

removing, in response to the sleeping aid value of the sleeping aid audio being less than a second threshold, a sleeping aid audio spectrum from which the sleeping aid audio is generated from the plurality of sleeping aid audio spectrums, to obtain a plurality of sleeping aid audio spectrums updated.

In some implementations, obtaining the sleep state of the user includes:

obtaining a plurality of physiological parameters of the user collected by a wearable device, in which the plurality of physiological parameters include at least one of: a number of times of turn-overs, a heart rate, a blood pressure, a respiratory rate or a head motion frequency; and

determining the sleep state of the user according to the plurality of physiological parameters.

According to a second aspect of the present disclosure, an apparatus for generating sleeping aid audio is provided, and includes:

at least one processor; and

a memory connected by communication with at least one processor; in which

the memory stores instructions that may be executed by at least one processor, and the instructions are executed by at least one processor, so that at least one processor may execute the methods described in the implementations in the above aspects.

According to a third aspect of the disclosure, a non-transitory computer readable storage medium that stores computer instructions is provided, and the computer instructions are executed by the computer to perform the method described in the implementations of the above aspects.

According to a fourth aspect of the disclosure, a computer program product is provided, including a computer program, the computer program is executed by the processor to perform a method of any one of the implementations of the above aspects.

It should be understood that the content described in this section is not intended to identify the key or important features of implementations of the disclosure, nor intended to limit the scope of the disclosure. Other features of the disclosure will be easily understood from the following description.

In an implementation of the present disclosure, a plurality of sleeping aid audio spectrums are obtained, and the plurality of sleeping aid audio spectrums are processed with a genetic algorithm, to obtain a sleeping aid audio spectrum chain, and the sleeping aid audio is generated according to the sleeping aid audio spectrum chain. Therefore, the sleeping aid audio spectrums can be used as genetic material to generate sleeping aid audio by using the genetic algorithm, which not only ensures an effect and reliability of sleeping aid audio, but also reduces a cost of sleeping aid audio.

Further, in an implementation of the disclosure, a plurality of reference audio spectrums are obtained, and each reference audio spectrum is identified with an identification model generated by training, to determine a sleeping aid value of each reference audio spectrum. Then, reference audio spectrums are determined from the m reference audio spectrums as the plurality of sleeping aid audio spectrums, each of the reference audio spectrums corresponds to a sleeping aid value greater than a first threshold. And the plurality of sleeping aid audio spectrums are processed based on the sleeping aid values corresponding to the plurality of sleeping aid audio spectrums with the genetic algorithm, to obtain the sleeping aid audio spectrum chain. The sleeping aid audio is generated according to the sleeping aid audio spectrum chain. Therefore, the sleeping aid audio is generated with the genetic algorithm by taking the audio spectrum with relatively high sleeping aid value as the genetic material, so as to further improve the effect and reliability of the generated sleeping aid audio.

Further, in an implementation of the disclosure, a plurality of sleeping aid audio spectrums are obtained, and the plurality of sleeping aid audio spectrums are processed with a genetic algorithm, to obtain a sleeping aid audio spectrum chain, and the sleeping aid audio is generated according to the sleeping aid audio spectrum chain. Sleep state parameters of a user are obtained, while the sleeping aid audio is played. The sleeping aid value of the sleeping aid audio is determined according to the sleep state parameters. In response to the sleeping aid value of the sleeping aid audio being less than a second threshold, a sleeping aid audio spectrum is removed from which the sleeping aid audio is generated from the plurality of sleeping aid audio spectrums, to obtain a plurality of sleeping aid audio spectrums updated. The sleeping aid audio corresponding to the user is generated by repeating above sleeping aid audio generation process based on the updated sleeping aid audio spectrum set. Thus, through the sleep state feedback of the user, the reliability and effectiveness of generating the sleeping aid audio are improved, perchildalized customization of the user is realized, and the sleeping aid audio with better sleep effect is generated scientifically and reliably.

It should be understood that the content described in this section is not intended to identify the key or important features of the implementations of the disclosure, nor to limit the scope of the disclosure. Other features of the disclosure will be easily understood through the following instructions.

### BRIEF DESCRIPTION OF DRAWINGS

Drawings are provided to better understand the present disclosure and are not intended to limit the present disclosure.

FIG. 1 is a schematic flowchart of a method for generating sleeping aid audio provided by the present disclosure.

FIG. 2 is a schematic flowchart of another method for generating sleeping aid audio provided by the present disclosure.

FIG. 3 is a schematic flowchart of another method for generating sleeping aid audio provided by the present disclosure.

FIG. 4 is a block diagram of an apparatus for generating a sleeping aid audio provided by the implementations of the present disclosure.

FIG. 5 is a block diagram of an electronic device provided by the implementations of the present disclosure.

### DETAILED DESCRIPTION

Implementations of the disclosure are described as below with reference to the drawings, which include various details of implementations of the disclosure to facilitate understanding and should be considered as merely example. Therefore, those skilled in the art should realize that various changes and modifications may be made to implementations described herein without departing from the scope and spirit of the disclosure. Similarly, for clarity and conciseness, descriptions of well-known functions and structures are omitted in the following descriptions.

The method for generating sleeping aid audio proposed in the present disclosure can be implemented by an apparatus for generating sleeping aid audio provided in the present disclosure, or by an electronic device provided in the present disclosure. The electronic device includes, but not limited to, a terminal device, such as a desktop computer or a tablet computer, or a server. The following describes the method for generating sleeping aid audio provided in the present disclosure, which is implemented by the apparatus for generating sleeping aid audio provided in the present disclosure, not as a limitation of the disclosure, and the apparatus for generating sleeping aid audio provided in the present disclosure is hereinafter referred to as “the apparatus”.

The following is a detailed description of the method, the apparatus, the computer device and storage medium for generating sleeping aid audio provided in the present disclosure.

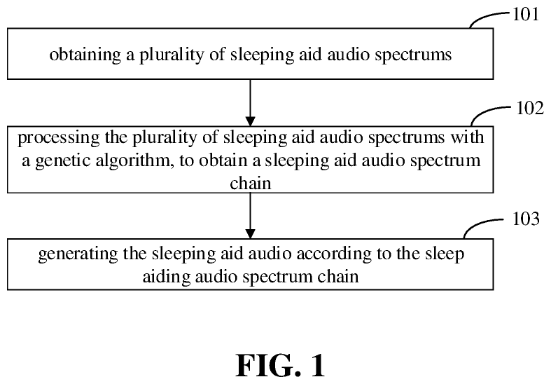

FIG. 1 is a schematic diagram of a method for generating sleeping aid audio according to the implementations of the present disclosure.

As illustrated in FIG. 1, in some implementations, the method for generating sleeping aid audio includes the following operations S101 to S103, which can be performed by at least one processor. The at least one processor can be, for example, part of an electronic device 500 described below in connection of FIG. 5. In one example, the electronic device 500 may be a terminal device that provides sleep-aiding audio to a user, e.g., a wearable device, a mobile phone, a tablet, etc. The electronic device 500 may be installed with a sleep-aiding audio application, or be in wireless communication with a terminal device that is installed with a sleep-aiding audio application. In another example, the electronic device 500 may be a server.

At S101, a plurality of sleeping aid audio spectrums are obtained.

The plurality of sleeping aid audio spectrums can be obtained in a variety of manners. In some implementations, a plurality of sleeping aid audios are acquired and then be processed by one or more processing, such as a Fourier variation processing and/or a selection operation, so as to obtain the plurality of sleeping aid audio spectrums. The plurality of sleeping aid audios may be created by a musician or a composer, or may be obtained from internet, a public library of sleeping aid audios, another electronic device, or the like. In other implementations, the plurality of sleep aid music spectrums can be obtained from one or more other electronic devices, internet, a public library of sleeping aid audios, or the like.

In one example, a plurality of audio spectrums are obtained, and the plurality of sleeping aid audio spectrums having a sleeping aid effect are selected from the plurality of audio spectrums. The audio spectrums obtained can be associated with audio characteristics including information representing the sleeping aid effect, and the sleeping aid audio spectrums can be chosen according to at least part of the audio characteristics of the audio spectrums. Alternatively, the sleeping aid effect of the audio spectrums can be determined by, for example, an identification model, and the sleeping aid audio spectrums are chosen according to the determined sleeping aid effect of the audio spectrums. For instance, an audio spectrum with a better sleeping aid effect is chosen to be the sleeping aid audio spectrum. For another instance, an audio spectrum with at least one audio characteristic satisfying certain condition is chosen to be the sleeping aid audio spectrum. The condition can be preset, or is set by the user, or is determined according to at least one of the user group profile and the particular sleeping aid purpose.

By combining melodies and music elements, sleeping aid audio may help people relieve stress and obtain physical and mental relaxation, so as to achieve a sleeping aid effect. Sleeping aid audio may be brain wave audio, binaural audio, audio played repeatedly at a fixed frequency, white noise or the like.

A sleeping aid audio spectrum refers to any type of audio spectrum with a sleeping aid effect and can be processed by performing one or more operations of combination, variation and/or the like to generate an audio.

At S102, the plurality of sleeping aid audio spectrums are processed with a genetic algorithm, to obtain a sleeping aid audio spectrum chain.

Specifically, after a sleeping aid audio spectrum set including the plurality of sleeping aid audio spectrums is obtained, one sleeping aid audio spectrum contained in the sleeping aid audio spectrum set can be used as a spectrum of the father generation for the genetic algorithm, so as to obtain the sleeping aid audio spectrum chain including at least one final sleeping aid audio spectrum. By applying the genetic algorithm, at least a part of the plurality of sleeping aid audio spectrums are taken as the audio spectrums of the first father generation and processed by at least one processing, such as a crossover processing and/or a mutation processing, to generate audio spectrums of the first child generation. At least a part of the audio spectrums of the first child generation are taken as the audio spectrums of the second father generation and processed by at least one processing, so as to obtain audio spectrums of the second child generation. The iteration goes on until a termination condition is satisfied, such as a number of the iteration reaches a preset maximum number, or it is determined that an optimal generation is obtained. For example, the audio spectrums of a father generation are processed by crossover processing; or, one or more audio spectrums of a father generation are processed by mutation processing, and the resulted one or more audio spectrums are then processed by cross-over processing with one or more audio spectrums of another father generation, so as to generate audio spectrums of a new generation. For another example, one or more audio spectrums of a father generation is chosen and included in the audio spectrums of its child generation.

It should be noted that there are many ways of performing the crossover and mutation processing. The crossover processing includes single point crossover, multipoint crossover, uniform crossover, arithmetic crossover, etc. The mutation processing includes uniform mutation, boundary mutation, Gaussian approximation mutation, etc.

By processing the plurality of sleeping aid audio spectrums with the genetic algorithm, audio spectrums of multiple generations can be obtained, and the sleeping aid audio spectrum chain is determined according to the audio spectrums of multiple generations. In one implementation, at least a part of the audio spectrums of the last generation are taken as the final sleeping aid audio spectrums included in the sleeping aid audio spectrum chain. In another implementation, the final sleeping aid audio spectrums are chosen from audio spectrums of at least one of the multiple generations according to their sleeping aid effects and/or other audio characteristics. For instance, the audio spectrums in the sleeping aid audio spectrum chain are chosen from the audio spectrums of the last N generations, where N>2. For another instance, the audio spectrums in the sleeping aid audio spectrum chain are chosen from audio spectrums of all generations.

At S103, the sleeping aid audio is generated according to the sleeping aid audio spectrum chain.

One or more processing is performed on the sleeping aid audio spectrum chain to obtain one or more sleeping aid audios. In one example, inverse Fourier transform is performed to decode at least one sleeping aid audio spectrum in the chain to obtain at least one sleeping aid audio.

It should be noted that, in the method for generating the sleeping aid audio, a noise signal in the sleeping aid audio is optionally removed according to a frequency preset by a user.

In the implementations of the present disclosure, a plurality of sleeping aid audio spectrums are obtained, and the plurality of sleeping aid audio spectrums are processed with the genetic algorithm, to obtain a sleeping aid audio spectrum chain. The sleeping aid audio is generated according to the sleeping aid audio spectrum chain. Therefore, the sleeping aid audio spectrums are used as genetic material to generate the sleeping aid audio with the genetic algorithm, which not only ensures the effect and reliability of sleeping aid audio, but also reduces the cost of sleeping aid audio.

In some implementations, at S102, the plurality of sleeping aid audio spectrums are processed with a genetic algorithm according to the sleeping aid effect of the sleeping aid audio spectrums, to obtain a sleeping aid audio spectrum chain. In one example, in the processing of the genetic algorithm, the sleeping aid effect of an audio spectrum is taken as a fitness parameter of the audio spectrum. In another example, in the processing of the genetic algorithm, the audio spectrums of a father generation are chosen according to their sleeping aid effects to produce audio spectrums of a next generation. In another example, in the processing of the genetic algorithm, the termination condition of the iteration is that a sleeping aid effect parameter (such as an average sleeping aid value) of the audio spectrums of the current generation achieves a preset threshold, or a ratio of the audio spectrums with a sleeping aid value reaches a preset threshold is higher than a preset ratio, or the like.

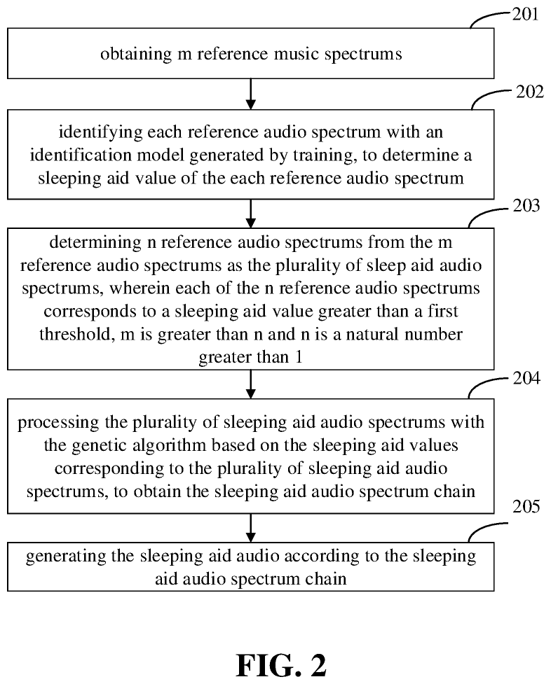

FIG. 2 is a schematic diagram of the method for generating sleeping aid audio according to some further implementations of the present disclosure.

As illustrated in FIG. 2, the method for generating the sleeping aid audio includes following operations S201 to S205, which can be performed by at least one processor. The at least one processor can be, for example, part of an electronic device 500 described below in connection of FIG. 5.

At S201, m reference audio spectrums are obtained, where m is a natural number greater than 1.

Optionally, the reference audio spectrums are obtained by processing original audio data of the reference audio. For example, for the original audio data of the reference audio, the original audio data is firstly segmented into audio segments with a fixed length, and the audio segments are then processed by Fourier transformation to obtain the reference audio spectrums corresponding to the audio segments.

In one example, the reference audio can be obtained from a public audio library, or be generated by a composer or a musician. The obtained reference audio can be associated with at least one audio characteristic, such as tune, melody, rhythm, tempo, type, genre, and/or information for indicating a sleeping aid effect of the audio. The sleeping aid effect of an audio can be indicated by at least one of a sleeping-aid score, or, a category indicator for indicating a sleeping-aid audio or a non-sleeping-aid audio. For example, the reference audio is associated with a category indicator for indicating it is a sleeping-aid audio or a non-sleeping-aid audio. For another example, the reference audio is associated with a sleeping-aid score for indicating the effectiveness of the audio in sleeping-aid, and optionally a reference audio with a sleeping-aid score lower than a preset threshold is determined to be a non-sleeping-aid audio. Since a sleeping aid audio may contain an audio segment with a relatively poor sleeping aid effect, while a non-sleeping-aid audio may contain an audio segment with relatively good sleeping aid effect. In order to better customize the sleeping aid audio for the user to help the user achieve high-quality sleep in the present disclosure, in some examples, a plurality types of reference audios are selected from massive audio data in advance, and the reference audio can be of any type.

In some implementations, the reference audio spectrums are the audio spectrums containing sleeping aid audio and non-sleeping-aid audio. For example, the audio spectrum of the non-sleeping-aid audio can include audio spectrum associated with music genres such as heavy metals, rock and/or hip-hop, etc., and the audio spectrum of sleeping aid audio can include audio spectrums associated with the effect of aiding a user's sleep such as brain wave audio, binaural audio, audio with a fixed repetition frequency and/or white noise, etc.

At S202, each reference audio spectrum is identified with an identification model generated by training, to determine a sleeping aid value of the each reference audio spectrum.

In some implementations, the identification model is obtained by training with samples labeled as a sleeping aid audio spectrum or a non-sleeping-aid audio spectrum. The identification model generated by training can identify the sleeping aid effect of each reference audio spectrum. The identification model optionally has an architecture of a convolutional neural network model or a recurrent neural network model.

Specifically, after a reference audio spectrum is inputted into the identification model, the sleeping aid effect of the reference audio spectrum is determined by the identification model. In some implementations, the output of the last layer of the identification model optionally includes a sleeping aid value representing the sleeping aid effect of the sleeping aid audio spectrum. A higher sleeping aid value indicates a better sleeping aid effect. The sleeping aid value is optionally used to determine whether the audio corresponding to the audio spectrum is a sleeping-aid audio or a non-sleeping-aid audio. For instance, if the sleeping aid value of an audio spectrum equals to or is higher than a threshold, the audio corresponding to the audio spectrum is determined to be a sleeping-aid audio, and if the sleeping aid value of an audio spectrum is less than the threshold, the audio corresponding to the audio spectrum is determined to be a non-sleeping-aid audio. In some other implementations, the output of the last layer of the identification model includes an identifier for representing a sleeping-aid audio spectrum or a non-sleeping-aid audio spectrum. The output of the last layer of the identification model may include other parameters representing the sleeping aid effect of the audio spectrum, and no limitation is set herein.

At S203, n reference audio spectrums from the m reference audio spectrums are determined as the plurality of sleeping aid audio spectrums, where each of the n reference audio spectrums corresponds to a sleeping aid value greater than a first threshold, m is greater than n, and n is a natural number greater than 1.

The first threshold is optionally a preset sleeping aid threshold. If the sleeping aid value corresponding to a reference audio spectrum is greater than the first threshold, it indicates that the sleeping aid effect of this reference audio spectrum is relatively good, and this reference audio spectrum is determined as the sleeping aid audio spectrum.

For example, there are four reference audio spectrums, that is, n is 4, and the four reference audio spectrums are spectrum a, spectrum b, spectrum c and spectrum d, respectively. Spectrums a, b, c and d correspond to the sleeping aid values of 16, 36, 84 and 77, respectively. If the preset first threshold is 75, spectrum c and spectrum d in the reference audio spectrums are determined as the sleeping aid audio spectrums.

At S204, the plurality of sleeping aid audio spectrums are processed with a genetic algorithm based on the sleeping aid values corresponding to the plurality of sleeping aid audio spectrums, to obtain the sleeping aid audio spectrum chain.

In some implementations, one or more target sleeping aid audio spectrums are selected from the plurality of sleeping aid audio spectrums according to their sleeping aid values, and the one or more target sleeping aid audio spectrums are processed by a crossover operation and/or a mutation operation to generate a plurality of first-child-generation sleeping aid audio spectrums.

In some implementations, the one or more target sleeping aid audio spectrums are spectrums with sleeping aid values equal to or higher than a threshold and are selected from the plurality of sleeping aid audio spectrums. By performing the crossover operation and/or the mutation operation on the target audio spectrums, the first-child-generation sleeping aid audio spectrums more suitable for sleeping aid are obtained. It should be noted that there are many ways of crossover operation and mutation operation. The crossover operation includes single point crossover, multipoint crossover, uniform crossover and/or arithmetic crossover, etc. The mutation operation includes uniform mutation, boundary mutation and/or Gaussian approximation mutation, etc.

Further, the first-child-generation sleeping aid audio spectrums are processed likewise to produce a second-child-generation sleeping audio spectrums. In some implementations, the sleeping aid values of the first-child-generation sleeping aid audio spectrums are determined, and one or more target first-child-generation sleeping aid audio spectrums are selected from the plurality of first-child-generation sleeping aid audio spectrums according to their sleeping aid values. The crossover operation and/or the mutation operation are then performed on the one or more target first-child-generation sleeping aid audio spectrums, so as to obtain the second-child generation sleeping audio spectrums, and so on, until a number of iterations performed reaches a preset value, such as, for example, until the N-child-generation sleeping audio spectrums are obtained, where N is a preset value. A sleeping aid audio spectrum chain is determined based on the target sleeping aid audio spectrums and the generated respective child generations of target sleeping aid audio spectrums.

It should be understood that, children audio spectrums, namely, the first-child-generation sleeping aid audio spectrums, with a high fitness parameter and/or a good sleeping aid effect are selectively retained by performing the crossover operation and/or the mutation operation. The target first-child-generation sleeping aid audio spectrums are the first-child-generation sleeping aid audio spectrums that better meets the requirements of sleeping aid. Based on the target first-child-generation sleeping aid audio spectrums, the crossover operation and/or the mutation operation are performed repeatedly to iterate and optimize the sleeping aid audio spectrums, so as to obtain the target-child sleeping aid audio spectrums, and thus the sleeping aid audio spectrum chain is obtained.

It should be noted that, in the method for generating the sleeping aid audio, when the genetic algorithm is used to process the sleeping aid audio spectrums, the sleeping aid values corresponding to the sleeping aid audio spectrums are used as fitness of the audio spectrums, and the one or more sleeping aid audio spectrums with sleeping aid values higher than a preset threshold are processed with the genetic algorithm. Thus, musical attributes of the sleeping aid audio generated tend to be more sleeping aid.

At S205, the sleeping aid audio is generated according to the sleeping aid audio spectrum chain.

It should be noted that, regarding the specific implementations of S205, the above implementations can be referred to, and will not be illustrated in detail herein.

In the implementations of the present disclosure, m reference audio spectrums are obtained, and each reference audio spectrum is identified with the identification model generated by training, to determine the sleeping aid value corresponding to the each reference audio spectrum. N reference audio spectrums are determined from the m reference audio spectrums as the plurality of sleeping aid audio spectrums, each of the n reference audio spectrums corresponds to a sleeping aid value greater than the first threshold. The plurality of sleeping aid audio spectrums are processed with the genetic algorithm based on the sleeping aid values corresponding to the plurality of sleeping aid audio spectrums, to obtain the sleeping aid audio spectrum chain. One or more sleeping aid audios are generated according to the sleeping aid audio spectrum chain. Therefore, the sleeping aid audio is generated with the genetic algorithm by taking the audio spectrum with relatively high sleeping aid value as the genetic material, so as to further improve the effect and reliability of the generated sleeping aid audios.

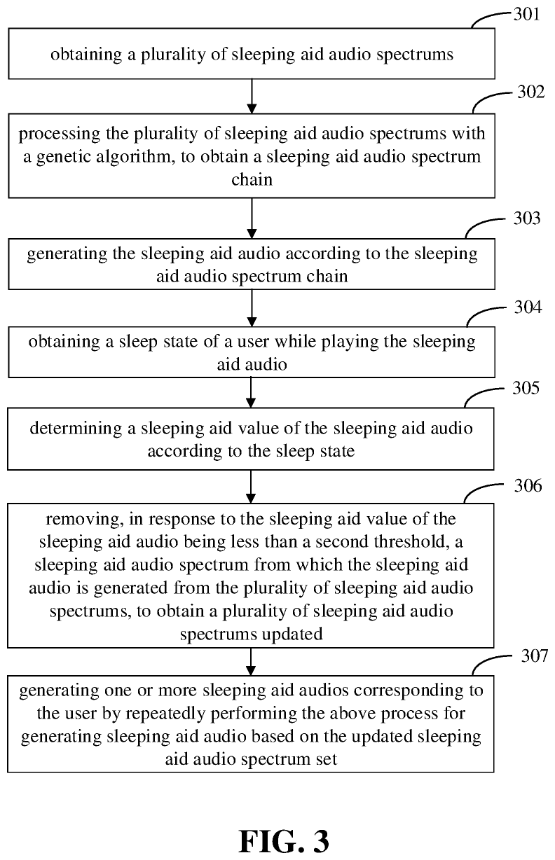

FIG. 3 is a schematic diagram of the method for generating a sleeping aid audio based on some further implementations of the present disclosure.

As illustrated in FIG. 3, the method for generating the sleeping aid audio includes the following operations S301 to S307, which can be performed by at least one processor. The at least one processor can be, for example, part of an electronic device 500 described below in connection of FIG. 5.

At S301, a plurality of sleeping aid audio spectrums are obtained.

At S302, the plurality of sleeping aid audio spectrums are processed with a genetic algorithm, to obtain a sleeping aid audio spectrum chain.

At S303, the sleeping aid audio is generated according to the sleeping aid audio spectrum chain.

It should be noted that, the above implementations can be referred to for the specific implementation process of S301, S302 and S303, which will not be illustrated in detail herein.

At S304, one or more sleep state parameters of the user are obtained while the sleeping aid audio is being played.

It should be noted that there are many ways to obtain the one or more sleep state parameters of the user. For example, a wearable device is used to collect a plurality of physiological parameters of the user, and the physiological parameters can include at least one of the following parameters: a number of times of turn-overs, a heart rate, a blood pressure, a respiratory rate or a head motion frequency. The one or more sleep state parameters of the user are then determined according to the physiological parameters.

The plurality of physiological parameters of the user can be collected by a wearable device, such as an electronic watch or a bracelet. Specifically, the physiological parameters include the number of times of turn-overs, the heart rate, the blood pressure, the respiratory rate and/or the head motion frequency, etc.

The one or more sleep state parameters are used for representing a sleep state of the user, such as deep sleep, sound sleep, light sleep, falling asleep, etc.

Optionally, one or more thresholds of the physiological parameters are preset, such as a count threshold of turn-over, a heart rate threshold, a blood pressure threshold, a respiratory rate threshold and/or a head motion frequency threshold. The sleep state of the user is then inferred by comparing the physiological parameters with the respective physiological parameter thresholds. For example, if all of the number of times of turn-overs, the head motion frequency of the user and the heart rate of the user are lower than corresponding preset thresholds when the user is listening to sleeping aid audio A, it indicates that the user is currently in a sound sleep state.

A mapping relationship between physiological parameters and the sleep state parameters is optionally preset, and the current sleep state parameters of the user are then determined according to the physiological parameters collected during a specific sleeping aid audio is being played.

In some implementations, the wearable device is configured to determine the sleep parameters of the user by monitoring spatial variations of a position and a posture of the user while the sleeping aid audio is being played.

It should be noted that, when a plurality of sleeping aid audios are played in sequence, one or more sleep state parameters of the user corresponding to each sleeping aid audio are optionally obtained.

At S305, the sleeping aid values of one or more sleeping aid audio spectrums for generating the one or more sleeping aid audios are determined according to the sleep state parameters.

The extent to which each sleeping aid audio affects the sleep state of the user, namely, the sleeping aid effect, is determined through the sleep state parameters.

Optionally, the sleep state parameters are inputted into a pre-trained sleeping aid identification network model, so that one or more sleeping aid values corresponding to the respective sleep state parameters corresponding to the one or more sleeping aid audios are determined. The sleeping aid identification network model can be realized by various deep neural networks (DNN). In some implementations, besides the sleep state parameters, the sleeping aid identification network model can also have other inputs, such as, for example, the sleeping aid audio, or the audio spectrum of the sleeping aid audio, or the subjective feedback or operation of the user, etc.

At S306, in response to the sleeping aid value of a sleeping aid audio being less than a second threshold, a sleeping aid audio spectrum for generating the sleeping aid audio is removed from the plurality of sleeping aid audio spectrums, to obtain a plurality of sleeping aid audio spectrums updated. Alternatively, in response to the sleeping aid value of a sleeping aid audio being less than a second threshold, a sleeping aid audio spectrum for generating the sleeping aid audio is removed from the sleeping aid audio spectrum chain, so as to obtain an updated sleeping aid audio spectrum chain.

It should be understood that, a sleeping aid effect of the sleeping aid audio is obtained by comparing the sleeping aid value of the sleeping aid audio with the second threshold. For example, if the sleeping aid value of the sleeping aid audio is higher than the second threshold, it indicates that the current sleeping aid audio has a high fitness parameter and a relatively good sleeping aid ability, and corresponding sleeping aid audio spectrum can be retained. For another example, if the sleeping aid value of the sleeping aid audio is lower than the second threshold, it indicates that the current sleeping aid audio has a relatively low fitness parameter and poor sleeping aid ability, and corresponding sleeping aid audio spectrum can be removed from the plurality of sleeping aid audio spectrums or from the sleeping aid audio spectrum chain. The sleeping aid audio spectrum set or the sleeping aid audio spectrum chain is updated by removing the sleeping aid audio spectrum which generates a sleeping aid audio with a sleeping aid value lower than the second threshold, so that the updated sleeping aid audio spectrum set or the updated sleeping aid audio spectrum chain is more suitable for the user.

At S307, one or more sleeping aid audios corresponding to the user are generated by repeatedly performing the above process S301-S303 for generating sleeping aid audio based on the updated sleeping aid audio spectrum set.

Specifically, by a continuous repetition of the process for generating the sleeping aid audio, the sleeping aid audio spectrum set is continuously adapted to sleep characteristics of the user, so as to rapidly adapt and meet the requirements of the user.

In some implementations of the present disclosure, a plurality of sleeping aid audio spectrums are obtained, and the plurality of sleeping aid audio spectrums are processed with a genetic algorithm, to obtain a sleeping aid audio spectrum chain. Sleep state parameters of a user are obtained while the sleeping aid audio is being played. The sleeping aid value of the sleeping aid audio is determined according to the sleep state parameters. In response to the sleeping aid value of one sleeping aid audio being less than a second threshold, a sleeping aid audio spectrum from which the one sleeping aid audio is generated is removed from the plurality of sleeping aid audio spectrums, so as to obtain a plurality of sleeping aid audio spectrums updated. One or more sleeping aid audios corresponding to the user are generated by repeatedly performing the above process for generating the sleeping aid audio based on the updated sleeping aid audio spectrum set. Therefore, through the sleep state feedback of the user, the reliability and effectiveness of generation of the sleeping aid audio are improved, personalized customization for the user is realized, and the sleeping aid audio with better sleep effect is generated scientifically and reliably.

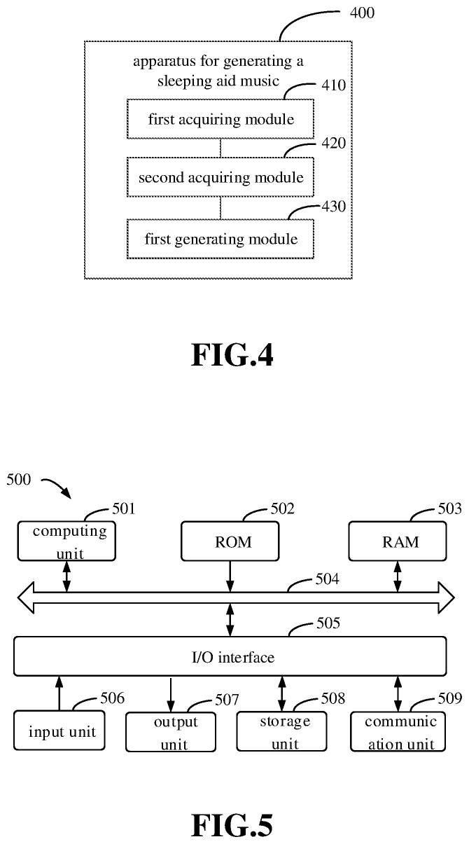

In order to realize the above implementations, some implementation of the present disclosure also provide an apparatus for generating sleeping aid audio. FIG. 4 illustrates a structural diagram of an apparatus for generating sleeping aid audio provided by some implementations of the present disclosure.

As illustrated in FIG. 4, the apparatus for generating the sleeping aid audio includes: a first acquiring module 410, a second acquiring module 420 and a first generating module 430.

The first acquiring module 410 is configured to obtain a plurality of sleeping aid audio spectrums.

The second acquiring module 420 is configured to process the plurality of sleeping aid audio spectrums with a genetic algorithm, to obtain a sleeping aid audio spectrum chain.

The first generating module 430 is configured to generate the sleeping aid audio according to the sleeping aid audio spectrum chain.

In some implementations, the first acquiring module 410 is specifically configured to: obtain m reference audio spectrums; identify each reference audio spectrum with an identification model generated by training, to determine a sleeping aid value of the each reference audio spectrum; and determine n reference audio spectrums from the m reference audio spectrums as the sleeping aid audio spectrums. Each of the n reference audio spectrums corresponds to a sleeping aid value greater than a first threshold, m is greater than n and n is a natural number greater than 1.

In some implementations, the second acquiring module 420 includes a processing unit, configured to process the plurality of sleeping aid audio spectrums with the genetic algorithm based on the sleeping aid values corresponding to the plurality of sleeping aid audio spectrums, to obtain the sleeping aid audio spectrum chain.

In some implementations, the processing unit is specifically configured to select one or more target sleeping aid audio spectrums to be processed from the plurality of sleeping aid audio spectrums based on the sleeping aid values corresponding to the plurality of sleeping aid audio spectrums; process the one or more target sleeping aid audio spectrums by performing at least one of a crossover operation and a mutation operation, to generate a plurality of first-child-generation sleeping aid audio spectrums; select one or more target first-child-generation sleeping aid audio spectrums from the plurality of first-child-generation sleeping aid audio spectrums based on the sleeping aid values corresponding to the plurality of first-child-generation sleeping aid audio spectrums; and preform, repeatedly until a number of operations performed reaches a preset value, at least one of the crossover operation and the mutation operation based on the one or more target first-child-generation sleeping aid audio spectrums, and determine the sleeping aid audio spectrum chain according to the one or more target sleeping aid audio spectrums and generated respective generations of target sleeping aid audio spectrums.

In some implementations, the first generating module 430 includes a first acquiring unit, a first generating unit and a second generating unit.

The first acquiring unit is configured to obtain a sleep state of a user while the sleeping aid audio is being played.

The first generating unit is configured to determine the sleeping aid value of the sleeping aid audio according to the sleep state.

The second generating unit is configured to remove a sleeping aid audio spectrum from which the sleeping aid audio is generated from the plurality of sleeping aid audio spectrums, to obtain a plurality of sleeping aid audio spectrums updated, in response to the sleeping aid value of the sleeping aid audio being less than a second threshold.

In some implementations, the first acquiring unit is configured to obtain a plurality of physiological parameters of a user collected by the wearable device, the plurality of physiological parameters include at least one of: a number of times of turn-overs, a heart rate, a blood pressure, a respiratory rate or a head motion frequency; and determine the sleep state of the user according to the plurality of physiological parameters.

In an implementation of the present disclosure, the apparatus may obtain a plurality of sleeping aid audio spectrums, and processes the plurality of sleeping aid audio spectrums with a genetic algorithm, to obtain a sleeping aid audio spectrum chain. The sleeping aid audio is generated based on the sleeping aid audio spectrum chain. Therefore, the sleeping aid audio spectrums may be used as genetic material to generate sleeping aid audio by using the genetic algorithm, which not only ensures an effect and reliability of sleeping aid audio, but also reduces a cost of sleeping aid audio.

According to an implementation of the present disclosure, a wearable device, a readable storage medium and a computer program product are provided.

FIG. 5 illustrates a schematic diagram of an electronic device 500 which may be used to implement implementations of the present disclosure. Electronic device aims to represent various forms of digital computers, such as laptop computers, desktop computers, worktables, personal digital assistants, servers, blade servers, large computers, and other suitable computers. An electronic device may also represent various forms of mobile devices, such as personal digital processors, cellular phones, smart phones, wearable devices and other similar computing devices. The components shown herein, their connections and relations, and their functions are merely examples, and are not intended to limit the implementation of the disclosure described and/or required herein.

As illustrated in FIG. 5, the device 500 includes a computing unit 501, which may execute various appropriate actions and processes based on the computer program stored in the read-only memory (ROM) 502 or a computer program loaded a random access memory (RAM) 503 from a storage unit 508. In the RAM 503, various programs and data required for operation of the device 500 may also be stored. The computing unit 501, the ROM 502 and the RAM 503 are connected to each other through a bus 504. An input/output (I/O) interface 505 is also connected to the bus 504.

Several components in the device 500 are connected to the I/O interface 505, and include: an input unit 506, for example, keyboard, mouse, etc. an output unit 507, for example, various types of displays, speakers; a storage unit 508, for example, a magnetic disk, an optical disk, etc.; and a communication unit 509, for example, a network card, a modem, a wireless communication transceiver, etc. The communication unit 509 allows a device 500 to exchange information/data through a computer network such as internet and/or various types of telecommunication networks and other devices.

The computing unit 501 may be various general-purpose and/or special-purpose processing components with processing and computing capacities. Some examples of the computing unit 501 include but are not limited to a central processing unit (CPU), a graphics processing units (GPU), various dedicated artificial intelligence (AI) computing chips, various computing units running a machine learning model algorithm, a digital signal processors (DSP), and any appropriate processor, controller, microcontroller, etc. The computing unit 501 executes various methods and processes as described above, for example, in some implementations, the method for generating the sleeping aid audio may be achieved as a computer software program, which is physically contained in a machine readable medium, such as a storage unit 508. In some implementations, a part or all of the computer program may be loaded and/or installed on the device 500 via a ROM 502 and/or a communication unit 509. When the computer program is loaded on a RAM 503 and executed by a computing unit 501, one or more blocks in the method for generating sleeping aid audio described above may be performed. In some implementations, the computing unit 501 may be configured to perform a method for generating a sleeping aid audio in any other appropriate ways (for example, virtue of a firmware).

Various implementation modes of the systems and technologies described above may be achieved in a digital electronic circuit system, a field programmable gate array (FPGA), an application-specific integrated circuit (ASIC), an application specific standard product (ASSP), a system-on-chip (SOC) system, a complex programmable logic device, a computer hardware, a firmware, a software, and/or combinations thereof. The various implementation modes may include: implementation in one or more computer programs, and the one or more computer programs may be executed and/or interpreted on a programmable system including at least one programmable processor, and the programmable processor may be a dedicated or a general-purpose programmable processor that may receive data and instructions from a storage system, at least one input apparatus, and at least one output apparatus, and transmit the data and instructions to the storage system, the at least one input apparatus, and the at least one output apparatus.

A computer code configured to execute a method in the present disclosure may be written with one or any combination of a plurality of programming languages. The programming languages may be provided to a processor or a controller of a general purpose computer, a dedicated computer, or other apparatuses for programmable data processing so that the function/operation specified in the flowchart and/or block diagram may be performed when the program code is executed by the processor or controller. A computer code may be performed completely or partly on the machine, performed partly on the machine as an independent software package and performed partly or completely on the remote machine or server.

In the context of the disclosure, a machine-readable medium may be a tangible medium that may contain or store a program intended for use in or in conjunction with an instruction execution system, apparatus, or device. A machine readable medium may be a machine readable signal medium or a machine readable storage medium. A machine readable storage medium may include but not limited to an electronic, magnetic, optical, electromagnetic, infrared, or semiconductor system, apparatus or device, or any appropriate combination thereof. A more specific example of a machine readable storage medium includes an electronic connector with one or more cables, a portable computer disk, a hardware, a random access memory (RAM), a read-only memory (ROM), an erasable programmable read-only memory (an EPROM or a flash memory), an optical fiber device, and a portable optical disk read-only memory (CDROM), an optical storage device, a magnetic storage device, or any appropriate combination of the above.

In order to provide interaction with the user, the systems and technologies described here may be implemented on a computer, and the computer has: a display apparatus for displaying information to the user (for example, a CRT (cathode ray tube) or an LCD (liquid crystal display) monitor); and a keyboard and a pointing apparatus (for example, a mouse or a trackball) through which the user may provide input to the computer. Other types of apparatuses may further be configured to provide interaction with the user; for example, the feedback provided to the user may be any form of sensory feedback (for example, visual feedback, auditory feedback, or tactile feedback); and input from the user may be received in any form (including an acoustic input, a speech input, or a tactile input).

The systems and technologies described herein may be implemented in a computing system including back-end components (for example, as a data server), or a computing system including middleware components (for example, an application server), or a computing system including front-end components (for example, a user computer with a graphical user interface or a web browser through which the user may interact with the implementation mode of the system and technology described herein), or a computing system including any combination of such back-end components, middleware components or front-end components. The system components may be connected to each other through any form or medium of digital data communication (for example, a communication network). Examples of communication networks include: a local area network (LAN), a wide area network (WAN), an internet and a blockchain network.

The computer system may include a client and a server. The client and server are generally far away from each other and generally interact with each other through a communication network. The relationship between the client and the server is generated by computer programs running on the corresponding computer and having a client-server relationship with each other. A server may be a cloud server, also known as a cloud computing server or a cloud host, is a host product in a cloud computing service system, to solve the shortcomings of large management difficulty and weak business expansibility existed in the conventional physical host and Virtual Private Server (VPS) service. A server further may be a server with a distributed system, or a server in combination with a block chain.

In an implementation of the present disclosure, a plurality of sleeping aid audio spectrums are obtained, and the plurality of sleeping aid audio spectrums are processed with a genetic algorithm, to obtain a sleeping aid audio spectrum chain. The sleeping aid audio is generated based on the sleeping aid audio spectrum chain. Therefore, the sleeping aid audio spectrums may be used as genetic material to generate sleeping aid audio by using the genetic algorithm, which not only ensures an effect and reliability of sleeping aid audio, but also reduces a cost of sleeping aid audio.

It should be understood that the above various forms of processes may be used to reorder, add or delete operations. For example, the operations recorded in the present disclosure may be executed in parallel, sequentially or in different orders. As long as the expected results of the technical scheme of the disclosure may be achieved, there is no limitation in this paper.

The above specific implementation does not constitute a limitation on the protection scope of the disclosure. Those skilled in the art should understand that various modifications, combinations, sub-combinations and substitutions may be made according to design requirements and other factors. Any modification, equivalent replacement, improvement, etc., made within the spirit and principle of implementations of the disclosure shall be included within the protection scope of the disclosure.

What is claimed is:

1. A method for generating a sleeping aid audio, comprising:
obtaining, by at least one processor, a plurality of sleeping aid audio spectrums;
processing, by the at least one processor, the plurality of sleeping aid audio spectrums with a genetic algorithm, to obtain a sleeping aid audio spectrum chain; and
generating, by the at least one processor, at least one sleeping aid audio according to the sleeping aid audio spectrum chain.

2. The method of claim 1, wherein obtaining, by the at least one processor, the plurality of sleeping aid audio spectrums comprises:
obtaining m reference audio spectrums;
processing, by the at least one processor, each reference audio spectrum with an identification model, to determine a sleeping aid value of the each reference audio spectrum; and
determining, by the at least one processor, n reference audio spectrums from the m reference audio spectrums as the plurality of sleeping aid audio spectrums, wherein each of the n reference audio spectrums corresponds to a sleeping aid value greater than a first threshold, m is greater than n, and n is a natural number greater than 1.

3. The method of claim 2, wherein processing, by the at least one processor, the plurality of sleeping aid audio spectrums with the genetic algorithm, to obtain the sleeping aid audio spectrum chain comprises:
processing, by the at least one processor, the plurality of sleeping aid audio spectrums with the genetic algorithm based on the sleeping aid values corresponding to the plurality of sleeping aid audio spectrums, to obtain the sleeping aid audio spectrum chain.

4. The method of claim 1, wherein processing, by the at least one processor, the plurality of sleeping aid audio spectrums with the genetic algorithm, to obtain the sleeping aid audio spectrum chain comprises:
selecting one or more target sleeping aid audio spectrums from the plurality of sleeping aid audio spectrums based on sleeping aid values corresponding to the plurality of sleeping aid audio spectrums;
processing the one or more target sleeping aid audio spectrums by performing at least one of a crossover operation and a mutation operation, to generate a plurality of first-child-generation sleeping aid audio spectrums;
selecting one or more target first-child-generation sleeping aid audio spectrums from the plurality of first-child-generation sleeping aid audio spectrums based on sleeping aid values corresponding to the plurality of first-child-generation sleeping aid audio spectrums;
preforming, repeatedly by the at least one processor until a termination condition is satisfied, at least one of the crossover operation and the mutation operation based on the one or more target first-child-generation sleeping aid audio spectrums, and
determining the sleeping aid audio spectrum chain according to the one or more target sleeping aid audio spectrums and generated respective child generations of target sleeping aid audio spectrums.

5. The method of claim 1, further comprising:
after generating, by the at least one processor, at least one sleeping aid audio according to the sleeping aid audio spectrum chain:
obtaining, by the at least one processor, a sleep state of a user while a sleeping aid audio of the at least one sleeping aid audio is being played;
determining, by the at least one processor, a sleeping aid value of the sleeping aid audio according to the sleep state; and
updating, by the at least one processor, according to the sleeping aid value of the sleeping aid audio, the plurality of sleeping aid audio spectrums, to obtain a plurality of sleeping aid audio spectrums updated.

6. The methods of claim 5, wherein obtaining, by the at least one processor, the sleep state of the user while the sleeping aid audio being played comprises:
obtaining a plurality of physiological parameters of the user collected by a wearable device, wherein the plurality of physiological parameters comprise at least one of: a number of times of turn-overs, a heart rate, a blood pressure, a respiratory rate or a head motion frequency; and
determining the sleep state of the user according to the plurality of physiological parameters.

7. An apparatus for generating a sleeping aid audio, comprising:
at least one processor; and
a memory communicatively connected to the at least one processor; wherein
the memory stores instructions executable by the at least one processor, and execution of the instructions by the at least one processor causes the at least one processor to perform the method of claim 1.

8. The apparatus of claim 7, wherein obtaining, by the at least one processor, the plurality of sleeping aid audio spectrums comprises:
obtaining m reference audio spectrums;
processing, by the at least one processor, each reference audio spectrum with an identification model generated by training, to determine a sleeping aid value of the each reference audio spectrum; and
determining, by the at least one processor, n reference audio spectrums from the m reference audio spectrums as the plurality of sleeping aid audio spectrums, wherein each of the n reference audio spectrums corresponds to a sleeping aid value greater than a first threshold, m is greater than n, and n is a natural number greater than 1.

9. The apparatus of claim 8, wherein processing, by the at least one processor, the plurality of sleeping aid audio spectrums with the genetic algorithm, to obtain the sleeping aid audio spectrum chain comprises:
processing, by the at least one processor, the plurality of sleeping aid audio spectrums with the genetic algorithm based on the sleeping aid values corresponding to the plurality of sleeping aid audio spectrums, to obtain the sleeping aid audio spectrum chain.

10. The apparatus of claim 9, wherein processing, by the at least one processor, the plurality of sleeping aid audio spectrums with the genetic algorithm based on the sleeping aid values corresponding to the plurality of sleeping aid audio spectrums, to obtain the sleeping aid audio spectrum chain comprises:
selecting one or more target sleeping aid audio spectrums to be processed from the plurality of sleeping aid audio spectrums based on the sleeping aid values corresponding to the plurality of sleeping aid audio spectrums;
processing the one or more target sleeping aid audio spectrums by performing at least one of a crossover operation and a mutation operation, to generate a plurality of first-child-generation sleeping aid audio spectrums;
selecting one or more target first-child-generation sleeping aid audio spectrums from the plurality of first-child-generation sleeping aid audio spectrums based on sleeping aid values corresponding to the plurality of first-child-generation sleeping aid audio spectrums; and
preforming, repeatedly by the at least one processor until a termination condition is satisfied, at least one of the crossover operation and the mutation operation based on the one or more target first-child-generation sleeping aid audio spectrums, and
determining the sleeping aid audio spectrum chain according to the one or more target sleeping aid audio spectrums and generated respective child generations of target sleeping aid audio spectrums.

11. The apparatus of claim 7, wherein the method performed by the at least one processor further comprises:
after generating, by the at least one processor, the at least one sleeping aid audio according to the sleeping aid audio spectrum chain:
obtaining, by the at least one processor, a sleep state of a user while a sleeping aid audio of the at least one sleeping aid audio is being played;
determining, by the at least one processor, a sleeping aid value of the sleeping aid audio according to the sleep state; and
updating, by the at least one processor, according to the sleeping aid value of the sleeping aid audio, the plurality of sleeping aid audio spectrums, to obtain a plurality of sleeping aid audio spectrums updated.

12. The apparatus of claim 11, wherein obtaining, by the at least one processor, the sleep state of the user while the sleeping aid audio of the at least one sleeping aid audio is being played comprises:
obtaining a plurality of physiological parameters of the user collected by a wearable device, wherein the plurality of physiological parameters comprise at least one of: a number of times of turn-overs, a heart rate, a blood pressure, a respiratory rate or a head motion frequency; and
determining the sleep state of the user according to the plurality of physiological parameters.

13. A non-transitory computer readable storage medium, having computer instructions stored thereon, wherein the computer instructions are configured to cause a computer to perform the method of claim 1.

14. The non-transitory computer readable storage medium of claim 13, wherein obtaining, by the at least one processor, the plurality of sleeping aid audio spectrums comprises:
obtaining m reference audio spectrums;
processing, by the at least one processor, each reference audio spectrum with an identification model generated by training, to determine a sleeping aid value of the each reference audio spectrum; and
determining, by the at least one processor, n reference audio spectrums from the m reference audio spectrums as the plurality of sleeping aid audio spectrums, wherein each of the n reference audio spectrums corresponds to a sleeping aid value greater than a first threshold, m is greater than n, and n is a natural number greater than 1.

15. The non-transitory computer readable storage medium of claim 14, wherein processing, by the at least one processor, the plurality of sleeping aid audio spectrums with the genetic algorithm, to obtain the sleeping aid audio spectrum chain comprises:
processing, by the at least one processor, the plurality of sleeping aid audio spectrums with the genetic algorithm based on the sleeping aid values corresponding to the plurality of sleeping aid audio spectrums, to obtain the sleeping aid audio spectrum chain.

16. The non-transitory computer readable storage medium of claim 15, wherein processing, by the at least one processor, the plurality of sleeping aid audio spectrums with the genetic algorithm based on the sleeping aid values corresponding to the plurality of sleeping aid audio spectrums, to obtain the sleeping aid audio spectrum chain comprises:
selecting one or more target sleeping aid audio spectrums to be processed from the plurality of sleeping aid audio spectrums based on the sleeping aid values corresponding to the plurality of sleeping aid audio spectrums;
processing the one or more target sleeping aid audio spectrums by performing at least one of a crossover operation and a mutation operation, to generate a plurality of first-child-generation sleeping aid audio spectrums;
selecting one or more target first-child-generation sleeping aid audio spectrums from the plurality of first-child-generation sleeping aid audio spectrums based on sleeping aid values corresponding to the plurality of first-child-generation sleeping aid audio spectrums; and
preforming, repeatedly by the at least one processor until a number of operations performed reaches a preset value, at least one of the crossover operation and the mutation operation based on the one or more target first-child-generation sleeping aid audio spectrums, and
determining the sleeping aid audio spectrum chain according to the one or more target sleeping aid audio spectrums and generated respective generations of target sleeping aid audio spectrums.

17. The non-transitory computer readable storage medium of claim 13, wherein the method performed by the computer further comprises:
after generating, by the at least one processor, the at least one sleeping aid audio according to the sleeping aid audio spectrum chain:
obtaining, by the at least one processor, a sleep state of a user while a sleeping aid audio of the at least one sleeping aid audio being played;
determining, by the at least one processor, a sleeping aid value of the sleeping aid audio according to the sleep state; and
removing, by the at least one processor, in response to the sleeping aid value of the sleeping aid audio being less than a second threshold, a sleeping aid audio spectrum from which the sleeping aid audio is generated from the plurality of sleeping aid audio spectrums, to obtain a plurality of sleeping aid audio spectrums updated.

18. The non-transitory computer readable storage medium of claim 17, wherein obtaining, by the at least one processor, the sleep state of the user while the sleeping aid audio being played comprises:
obtaining a plurality of physiological parameters of the user collected by a wearable device, wherein the plurality of physiological parameters comprise at least one of: a number of times of turn-overs, a heart rate, a blood pressure, a respiratory rate or a head motion frequency; and
determining the sleep state of the user according to the plurality of physiological parameters.

19. A computer program product comprising a computer program, wherein during execution of the computer program by at least one processor, the method of claim 1 is performed.

20. The computer program product of claim 19, wherein the method performed by the at least one processor further comprises:
after generating, by the at least one processor, the at least one sleeping aid audio according to the sleeping aid audio spectrum chain:
obtaining, by the at least one processor, a sleep state of a user while a sleeping aid audio of the at least one sleeping aid audio being played;
determining, by the at least one processor, a sleeping aid value of the sleeping aid audio according to the sleep state; and
removing, by the at least one processor, in response to the sleeping aid value of the sleeping aid audio being less than a second threshold, a sleeping aid audio spectrum from which the sleeping aid audio is generated from the plurality of sleeping aid audio spectrums, to obtain a plurality of sleeping aid audio spectrums updated.
